(19) United States
(12) Patent Application Publication
(10) Pub. No.: US 2026/0227345 A1
(43) Pub. Date: Aug. 6, 2026

(54) MEASUREMENT ASSEMBLY FOR DETERMINING A PROPERTY OF A MULTIPHASE FLOWABLE MEDIUM

(71) Applicant: Endress+Hauser Flowtec AG, Reinach (CH)

(72) Inventors: Günther Bähr, Therwil (CH); Stefan Pflüger, München (DE)

(21) Appl. No.: 19/138,203

(22) PCT Filed: Dec. 11, 2023
(86) PCT No.: PCT/EP2023/085158

§ 371 Date: Jun. 12, 2025

(30) Foreign Application Priority Data
Dec. 16, 2022 (DE) 10 2022 133 708.1

(51) Int. Cl. G01N 22/04

(52) U.S. Cl. CPC G01N 22/04

(57) ABSTRACT

A measurement assembly for determining a property of a multiphase flowable medium comprises a measuring tube for conveying the medium and a first microwave antenna device arranged in a first antenna receptacle of the measuring tube. The first microwave antenna device is designed to emit a first microwave signal in a first main radiation direction and a second microwave signal in a second main radiation direction different from the first. A measurement circuit connected to the first microwave antenna device includes a high-frequency generator for supplying the first microwave signal and the second microwave signal. The measurement circuit is configured to determine the property of the medium based on the measured first microwave signal, and the measurement circuit is further configured to detect an inhomogeneity in the measuring tube based on the measured first microwave signal and the measured second microwave signal.

The invention relates to a measurement assembly for determining a property of a multiphase flowable medium by means of a microwave antenna device.

It is possible, by means of microwaves, to determine the physical quantities of permittivity and loss factor of a medium in a process line. From these two variables—measured either at one or over many different frequencies—it is possible to draw conclusions regarding application-specific parameters, for example the proportion of water in a mixture of water and other non-polar or weakly polar components or a solid content in a liquid medium.

The established transmission/reflection measurement is described in L. F. Chen, C. K. Ong, C. P. Neo, V. V. Varadan, V. K. Varadan—“Microwave Electronics, Measurement and Materials Characterization,” John Wiley & Sons Ltd., 2004. For this purpose, the microwave signal interfaces at two different positions at the medium in a container or measuring tube, the scatter parameters (transmission and optionally reflection) are measured between these interface structures, and the mentioned physical properties of the medium are calculated from the measured scatter parameters.

WO 2018 121927 A1 teaches a measuring assembly for analyzing properties of a flowing medium by means of microwaves. In addition to the microwave antennas, the measuring assembly has an electrically insulating lining layer on the inner peripheral surface of the measuring tube. This lining layer forms a dielectric waveguide via which at least part of the one microwave signal can travel from a first microwave antenna to a second microwave antenna. One application for such a measuring assembly is the determination of the proportions of solids in the liquid medium being conveyed. WO 2021/099152 A1 teaches a microwave antenna which has a front section that comes into contact with the medium and via which the excitation signal is emitted into the medium.

Processes in which solid components are present in the medium suffer from the deposition of solids on the inner wall of the measuring tube and sedimentation. WO 2016/075367 A1 discloses a method for detecting deposits on the utilized microwave antennas, which come into contact with the medium. For this purpose, in addition to the conventional two opposite microwave antennas, at least one further microwave antenna is used which is arranged on the measuring tube in such a way that two measurement paths of different lengths result. The presence of a coating is determined on the basis of the time between the emission and reception of the microwave signals (time-of-flight) for the two measuring paths. The disadvantage of the solution is that at least a third microwave antenna is required.

The object addressed by the invention is to provide an alternative.

The object is achieved by the measurement assembly according to claim 1 and the method according to claim 16.

The measurement assembly according to the invention for determining a property of a multiphase flowable medium comprises:

a measuring tube for conveying the medium,
wherein the measuring tube has a first antenna receptacle,
   a first microwave antenna device which is arranged in the first antenna receptacle,
wherein the first microwave antenna device is designed so as to emit a first microwave signal and a second microwave signal,
wherein the first microwave signal has a first main radiation direction, and the second microwave signal has a second main radiation direction differing from the first main radiation direction; and
   a measurement circuit,
wherein the measurement circuit is in connection with the first microwave antenna device,
wherein the measurement circuit has a high-frequency generator for supplying the first microwave antenna device with the first microwave signal and the second microwave signal,
wherein the measurement circuit is configured to determine the property of the medium at least on the basis of the measured first microwave signal,
wherein the measurement circuit is configured to detect any inhomogeneity in the measuring tube on the basis of the measured first microwave signal and the measured second microwave signal.

In contrast to WO 2016/075367 A1, the two measurement paths are realized by two microwave signals having different main radiation directions, and not by two pairs of microwave antennas. This can already be achieved by using precisely one microwave antenna which is configured not only to emit the first microwave signal and the second microwave signal into the measuring tube, but also to receive them once they have passed through the medium.

The advantage of this is that an additional microwave antenna device is not required in addition to the number of microwave antenna devices already sufficient for determining the property of the multiphase medium. Each additional microwave antenna device requires a corresponding antenna receptacle on the measuring tube, which forms a potential leak point. In addition, each microwave antenna device is associated with additional costs. Furthermore, it is also more complex to provide electronics that can process more than two signal paths.

Advantageous embodiments of the invention are the subject matter of the dependent claims.

One embodiment provides that the second main radiation direction is selected such that a propagation direction of the microwave signal changes between the emission and the reception of the second microwave signal.

The second main radiation direction in which the second microwave signal is emitted is preferably selected such that the propagation direction changes at least once during the propagation of the second microwave signal through the medium. The change in the propagation direction results from the reflection of the second microwave signal at the inner wall of the measuring tube and/or at a medium boundary.

One embodiment provides that the first main radiation direction points radially into the measuring tube.

One embodiment provides that the first microwave antenna device is in particular mechanically pivotable.

The generation of the two microwave signals having different main radiation directions can be realized by designing the first microwave antenna to be mechanically pivotable. The first microwave antenna device accordingly comprises a mechanical pivoting device which is configured to mechanically align the microwave antenna. This allows the two main radiation directions to be set. The pivoting device can, for example, comprise at least one piezoelectric element which can be electrically actuated and which interacts with the microwave antenna in such a way that the main radiation direction of the microwave signal emitted by the microwave antenna can be controlled via the voltage applied to the piezoelectric element. Therefore, an electromechanical solution can be realized for pivoting the microwave antenna device.

The pivoting allows for operation in at least two modes. On the one hand, there is standard mode for optimal determination of the solid content. On the other hand, there is a diagnostic mode for detecting deposits or partial filling.

One embodiment provides that the first microwave antenna device is designed to electronically pivot the first main radiation direction of the second microwave signal.

The generation of the two microwave signals having different main radiation directions can be realized by designing the first microwave antenna device to be electronically pivotable. Microwave antenna devices are known which are suitable for generating microwave signals having different main radiation directions.

An example of this would be the phased array antenna, which is a phase-controlled antenna array with strong directivity. It achieves a bundling of the radiation energy and thus electronic beam pivoting through the array and connection of individual transmission elements (at least two microwave antennas). The antenna array uses the phase shift of the individual transmission elements arranged in the array to achieve a bundling of the energy as a result of interference of the individually generated microwave signals. The transmission energy is amplified in the desired main field direction, while the unwanted directions are eliminated by destructive interference.

One embodiment provides that the two microwave signals are fed into the medium simultaneously, in particular due to the excitation of two modes of the microwave antenna device by means of an excitation signal having at least two different excitation frequencies or by means of at least two excitation signals each having a different excitation frequency.

The waveguide of the microwave antenna can be designed in such a way that not only one mode with exactly one characteristic field pattern propagates therein in the desired frequency band, but at least two modes are formed. These can be excited by different frequencies that result in microwave signals which have different main radiation directions. It is therefore possible to feed either one excitation signal having at least two different frequencies or two excitation signals with different frequencies into the waveguide at different times in order to generate the two microwave signals. It would be possible to separate the two measured microwave signals if the two frequencies were chosen to be adequately different.

One embodiment provides that the measurement assembly further comprises:

a second microwave antenna device which is arranged in a second antenna receptacle of the measuring tube and is in particular oriented opposite the first antenna receptacle,
wherein the second microwave antenna device is configured to measure the first microwave signal and the second microwave signal.

One embodiment provides that the second microwave antenna device is designed mechanically pivotable, or
wherein the second microwave antenna device is designed to electronically pivot the second main radiation direction of the second microwave signal.

The advantage of limiting the number of microwave antenna devices to exactly two is a more compact, and in particular shorter, design of the measurement assembly.

One embodiment provides that the first main radiation direction is directed along the shortest connection between the first microwave antenna device and the second microwave antenna device.

One embodiment provides that the measurement assembly comprises a position sensor which is configured to determine the current orientation of the measuring tube and/or the first microwave antenna device relative to Earth,
wherein the measurement circuit is configured to take into account the current orientation of the measuring tube when detecting the inhomogeneity, in particular when determining a cause of the inhomogeneity.

By means of the position sensor, it is possible to distinguish whether any determined inhomogeneity is caused by the formation of a deposit or by a partial filling of the measuring tube.

One embodiment provides that the measurement circuit is configured to determine a first measured value for a solids content of the medium from the measured first microwave signal,
wherein the measurement circuit is configured to determine a second measured value for the solids content from the measured second microwave signal,
wherein the first measured value and the second measured value are included when detecting the inhomogeneity, in particular when determining a cause of the inhomogeneity.

One embodiment provides that the first microwave antenna device is configured to emit a third microwave signal having a third main radiation direction,
wherein the third main radiation direction of the third microwave signal differs from the first main radiation direction and the second main radiation direction,
wherein the measurement circuit is configured to detect any inhomogeneity in the measuring tube on the basis of the first microwave signal, the second microwave signal and a third microwave signal.

One embodiment provides that a measuring tube longitudinal section running through the first microwave antenna device divides the measuring tube into a first measuring tube section and a second measuring tube section,
wherein the second main radiation direction points into the first measuring tube section, and the third main radiation direction points into the second measuring tube section.

One embodiment provides that the inhomogeneity includes an in particular asymmetric deposit formation, partial filling of the measuring tube, and/or sedimentation formation.

One embodiment provides that the first main radiation direction and the second main radiation direction lie in a common measuring tube cross section intersecting at least the first microwave antenna device.

One embodiment provides that the second main radiation direction, in particular the second main radiation direction and the third main radiation direction, has a directional component that points in the longitudinal direction of the measuring tube.

If the second main radiation direction has a directional component that points in the longitudinal direction of the measuring tube, additional information about the properties of the medium outside the measuring cross section can be determined. Given a vertical orientation of the measurement assembly in the process line and partial filling of the measuring tube, the partial filling itself can be detected.

The method according to the invention for determining any inhomogeneity in a measuring tube by means of a measurement assembly according to any one of the preceding claims comprises the method steps of:

emitting a first microwave signal having a first main radiation direction into the measuring tube by means of a first microwave antenna device;
receiving the first microwave signal after emission, in particular by means of a second microwave antenna device,
wherein the received first microwave signal results at least from the emitted first microwave signal and its interaction with the medium;
   emitting a second microwave signal having a second main radiation direction into the measuring tube by means of the first microwave antenna device,
wherein the first main radiation direction differs from the second main radiation direction;
   receiving the second microwave signal after emission, in particular by means of a second microwave antenna device,
wherein the received second microwave signal results at least from the second transmitted microwave signal and its interaction with the medium,
wherein the second microwave signal is reflected at least once on an inner lateral surface of the measuring tube so that the second propagation direction spatially changes at least once between the emission of the second microwave signal and the reception of the second microwave signal;
   detecting whether any inhomogeneity exists in the measuring tube depending on the received first microwave signal and the second microwave signal.

One embodiment of the method according to the invention comprises:

emitting a third microwave signal having a third main radiation direction into the measuring tube by means of the first microwave antenna device;
receiving the third microwave signal after emission, in particular by means of the second microwave antenna device,
wherein the received third microwave signal results at least from the emitted third microwave signal and its interaction with the medium;
   detecting whether any inhomogeneity exists in the measuring tube depending on the received first microwave signal, second microwave signal and third microwave signal.

The invention is explained in greater detail with reference to the following figures. in which:

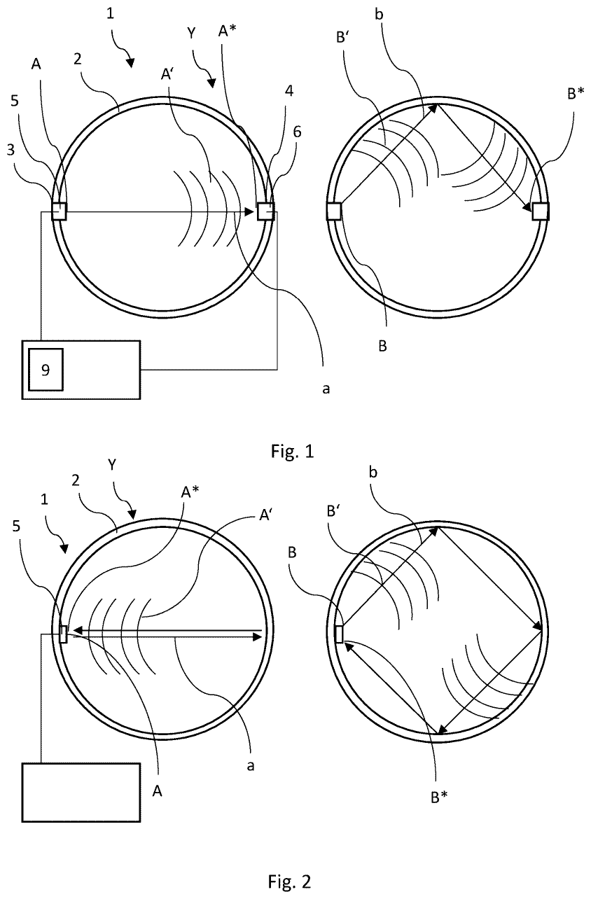

FIG. 1: is a cross section through one embodiment of the measurement assembly according to the invention;

FIG. 2: is a cross section through another embodiment of the measurement assembly according to the invention;

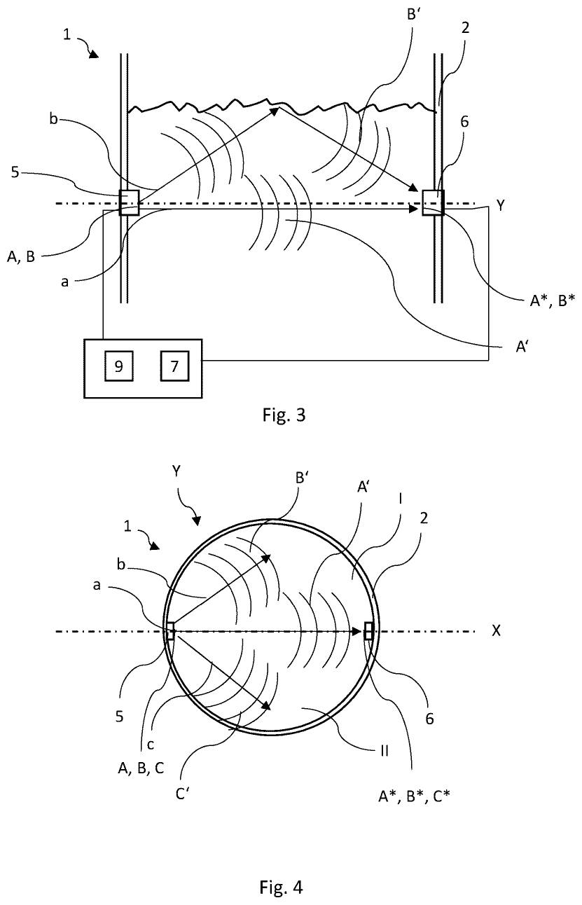

FIG. 3: is a longitudinal section through another embodiment of the measurement assembly according to the invention; and

FIG. 4: is a cross section through another embodiment of the measurement assembly according to the invention.

FIG. 1 shows a cross section through one embodiment of the measurement assembly 1 according to the invention for determining a property of a multiphase flowable water-based medium. The measurement assembly 1 comprises a measuring tube 2 for conducting the substantially aqueous medium with solid components. In the embodiment shown here, the measuring tube 2 is cylindrical and has a metal inner measuring tube wall. According to the invention, other measuring tube shapes are also provided which are homeomorphic to the cylinder geometry.

A first antenna receptacle 3 in the form of a measuring tube opening is formed in the measuring tube 2. The measuring tube 2 can be made of metal, and the measuring tube opening can be realized by a bore. A first microwave antenna device 5 is arranged medium-tight in the first antenna receptacle 3. The first microwave antenna device 5 has at least one microwave antenna. Furthermore, the first microwave antenna device 5 can have a holder for the at least one microwave antenna which is configured to immovably hold the microwave antenna in the measuring tube opening. The first microwave antenna device 5 is designed to emit a first microwave signal A and a second microwave signal B. The two microwave signals substantially differ in terms of the given propagation direction. The first microwave signal A has a first main radiation direction a in which the microwave signal A is radiated into the medium, and the second microwave signal B has a second main radiation direction b which differs from the first main radiation direction a and in which the second microwave signal B is radiated into the medium. The two microwave signals pass through the measuring tube and interact with the medium flowing through the measuring tube (see the first microwave signal A′ and second microwave signal B′). The second microwave signal B′ is reflected once at the inner wall of the measuring tube. The second main radiation direction b is selected such that a propagation direction of the microwave signal changes between the emission of the second microwave signal B and the reception of the second microwave signal B*. The first main radiation direction a and the second main radiation direction b lie in a common measuring tube cross section Y of the measuring tube 2 intersecting at least the first microwave antenna device 5.

The first microwave antenna device 5 is suitable for beam pivoting. This can be done by means of a mechanical or electromechanical pivoting device so that the first microwave antenna device 5 is designed mechanically pivotable. Alternatively, the first microwave antenna device 5 can be designed to electronically pivot the microwave signal such that a first main radiation direction a and a second main radiation direction b are provided. For this purpose, the first microwave antenna device 5 comprises at least two microwave antennas arranged in an array. The first microwave antenna device 5 is configured to shift the phase position at the microwave antenna such that different main radiation directions can be set as a result of interference and bundling.

The illustrated measurement assembly 1 further has a second microwave antenna device 6 which is arranged in a second antenna receptacle 4 of the measuring tube 2, in particular oriented opposite the first antenna receptacle 3. As a result, the first main radiation direction a is directed along the shortest connection between the first microwave antenna device 5 and the second microwave antenna device 6.

The second microwave antenna device 6 is configured to receive and measure the first microwave signal A* and the second microwave measurement signal B*. The first microwave signal A′ takes the direct path to the second microwave antenna device 6, i.e. it is not reflected. The first microwave signal A′ interacts with the medium on its way to the second microwave antenna device 6 so that the measured first microwave signal A* contains information about the medium. The same also applies to the second microwave signal B′ and the measured second microwave signal B*. The second microwave antenna device 6 can also be designed mechanically pivotable. Alternatively, the second microwave antenna device 6 can be designed such that the second main radiation direction b of the second microwave signal B can be electronically pivoted.

The measurement assembly 1 further has a measurement circuit 9 which is connected to the first microwave antenna device 5 and the second microwave antenna device 6. The measurement circuit 9 has a high-frequency generator for generating the microwave signals. The high-frequency generator generates a first microwave signal A which is fed into the medium or into the interior of the measuring tube via the first microwave antenna device 5. The second microwave signal B is also generated by the high-frequency generator and fed into the medium or the interior of the measuring tube via the first microwave antenna device 5. The measurement circuit 9 is configured to measure the first microwave signal A* and the second microwave signal B* passing through the interior of the measuring tube at the second microwave antenna device 6. The first microwave signal A and the second microwave signal B propagate through the medium. This results in an interaction between the microwave signals A, B and the medium, which changes the microwave signals A, B.

The measurement circuit 9 is further configured to determine a first measured value for a solids content of the medium from the measured first microwave signal A* and a second measured value for the solids content from the measured second microwave signal B*. Using an algorithm, the inhomogeneity is then detected on the basis of the first measured value and the second measured value. Inhomogeneities may be in particular asymmetric deposit formation, partial filling of the measuring tube 2, and/or sedimentation formation.

FIG. 2 shows a cross section through another embodiment of the measurement assembly 1 according to the invention. The depicted measurement assembly substantially differs from the embodiment in FIG. 1 in that the measurement assembly 1 only has precisely one microwave antenna device. This is configured to emit a first microwave signal A having a first main radiation direction a, which points, in particular radially, into the measuring tube 2. The generated first microwave signal A propagates along the measuring tube diameter (becomes the first microwave signal A′ upon interaction with medium) and is reflected back on the opposite side of the first microwave antenna device 5. The first microwave signal A* is measured at the first microwave antenna device 5. The resulting measurement path corresponds to twice the inner diameter of the measuring tube 2. The first microwave antenna device 5 is further configured to emit a second microwave signal B that has a second main field direction b in which the microwave signal B is emitted and which differs from the first main field direction a. The second main field direction b is selected such that the second microwave signal B is reflected three times at the inner wall and propagates back to the first emitting microwave antenna device 5 where it is measured. Alternatively, the second main field direction b can be selected such that the microwave signal is reflected only twice at the inner wall before it is detected by the first microwave antenna device 5. Alternatively, the second main field direction b can also be selected such that the microwave signal is reflected more than three times at the inner wall before it is detected by the first microwave antenna device 5.

FIG. 3 shows a longitudinal section through another embodiment of the measurement assembly 1 according to the invention. The measurement assembly 1 has a vertical mounting orientation, i.e. the longitudinal axis of the measurement assembly 1 or the measuring tube intersects the Earth's surface substantially vertically. A potential application is the use of the measurement assembly 1 in a riser pipe. In such applications, the measuring tube may be partially filled if the medium to be conveyed comes to a standstill and (partial) backflow of the medium occurs. In order to detect the installation position, the measurement assembly 1 has a position sensor 7 which is configured to determine the current orientation of the measuring tube 2 and/or the first microwave antenna device 5 relative to Earth or Earth's gravitational pull. The determined orientation is then included in the detection of the inhomogeneity, in particular when determining a cause for the inhomogeneity. As indicated, the position sensor 7 can be arranged in a common module together with the measurement circuit 9. Alternatively, the position sensor 7 can also be arranged in the sensor itself.

The measurement assembly 1 has a first microwave antenna device 5 and a second microwave antenna device 6. The first microwave antenna device 5 is configured to radiate a first microwave signal A radially into the measuring tube. The first main radiation direction a lies substantially in the cross-sectional plane Y which intersects the two microwave antenna devices and is oriented perpendicularly to the longitudinal axis of the measuring tube. Furthermore, the first microwave antenna device 5 is suitable for emitting a second microwave signal B having a second main radiation direction b. The second main radiation direction b has a directional component that points in the longitudinal direction of the measuring tube 2. The second main radiation direction b thus points out of the cross-sectional plane Y. If the measuring tube or the process line is partially filled, the second microwave signal B′ propagating through the medium is reflected at the medium boundary and measured by means of the second microwave antenna device 6. The measurement circuit 9 is configured to infer a partial filling of the measuring tube on the basis of the measured microwave signal B* and the determined orientation.

FIG. 4 shows a cross section through another embodiment of the measurement assembly 1 according to the invention. The embodiment in FIG. 4 substantially differs from the embodiment in FIG. 1 in that the first microwave antenna device 5 is also configured to emit a third microwave signal C having a third main radiation direction c. The third main radiation direction c of the third microwave signal C differs from the first main radiation direction a and the second main radiation direction b. A measuring tube longitudinal section X running through the first microwave antenna device 5 divides the measuring tube 2 into a first measuring tube section I and a second measuring tube section II. In the depicted embodiment, the longitudinal axis of the measuring tube is also located in the longitudinal section X of the measuring tube. With regard to the depicted embodiment, it is essential that the second main radiation direction b points into the first measuring tube section I, and the third main radiation direction c points into the second measuring tube section II. The measurement circuit 9 is configured to detect any inhomogeneity in the measuring tube 2 on the basis of the first microwave signal A*, the second microwave signal B*, and the third microwave signal C* measured at the second microwave antenna device 6. Especially when the measurement assembly 1 is installed horizontally, sedimentation can form in the second measuring tube section II. Sedimentation differs from deposit formation in that the solids of the medium are not deposited substantially uniformly over the entire inner circumference of the inner wall, but exclusively in a lower portion of the measuring tube. The third microwave signal C′ propagates through the medium and also through the sediment. By means of the measured third microwave signal C* it is therefore possible to distinguish whether sedimentation or deposit formation has occurred. If uniform deposit formation has occurred along the circumference of the measuring tube, the measured second microwave signal B* substantially matches the measured third microwave signal C*. If sedimentation has occurred, the two measured microwave signals B* and C* differ more from one another.

### LIST OF REFERENCE SIGNS

measurement assembly 1
measuring tube 2
first antenna receptacle 3
second antenna receptacle 4
first microwave antenna device 5
second microwave antenna device 6
position sensor 7
measuring circuit 9
first microwave signal A
first main radiation direction a
second microwave signal B
second main radiation direction b
third microwave signal C
third main radiation direction c
measured first microwave signal A*
measured second microwave signal B*
measured third microwave signal C*
first measuring tube section I
second measuring tube section II
measuring tube longitudinal section X
measuring tube cross section Y

1-16. (canceled)

17. A measurement assembly for determining a property of a multiphase, flowable medium, comprising:
a measuring tube for conveying the medium, wherein the measuring tube has a first antenna receptacle;
a first microwave antenna device arranged in the first antenna receptacle, wherein the first microwave antenna device is designed to emit a first microwave signal having a first main radiation direction and a second microwave signal having a second main radiation direction that differs from the first main radiation direction; and
a measurement circuit connected to the first microwave antenna device,
wherein the measurement circuit has a high-frequency generator for supplying the first microwave antenna device with the first microwave signal and the second microwave signal,
wherein the measurement circuit is configured to determine the property of the medium at least on the basis of a measured first microwave signal, and
wherein the measurement circuit is further configured to detect any inhomogeneity in the medium in the measuring tube on the basis of the measured first microwave signal and a measured second microwave signal.

18. The measurement assembly according to claim 17,
wherein the second main radiation direction is selected such that a propagation direction of the second microwave signal changes between an emission and a reception of the second microwave signal.

19. The measurement assembly according to claim 17,
wherein the first main radiation direction points radially into the measuring tube and/or in a direction of a longitudinal axis of the first microwave antenna device.

20. The measurement assembly according to claim 17,
wherein the first microwave antenna device is configured to be mechanically pivotable.

21. The measurement assembly according to claim 17,
wherein the first microwave antenna device is configured to electronically pivot the second main radiation direction of the second microwave signal.

22. The measurement assembly according to claim 17, further comprising:
a second microwave antenna device that is arranged in a second antenna receptacle of the measuring tube orientated opposite the first antenna receptacle,
wherein the second microwave antenna device is configured to measure the first microwave signal and the second microwave measurement signal.

23. The measurement assembly according to claim 22,
wherein the second microwave antenna device is configured to be mechanically pivotable, or
wherein the second microwave antenna device is configured to be electronically pivotable.

24. The measurement assembly according to claim 23,
wherein the first main radiation direction is directed along a shortest connection between the first microwave antenna device and the second microwave antenna device.

25. The measurement assembly according to claim 17, further comprising:
a position sensor that is configured to determine a current orientation of the measuring tube and/or the first microwave antenna device with respect to a gravitational force,
wherein the measurement circuit is further configured to take into account the current orientation of the measuring tube when detecting the inhomogeneity.

26. The measurement assembly according to claim 17,
wherein the measurement circuit is configured to determine a first measured value for a solids content of the medium from the measured first microwave signal,
wherein the measurement circuit is configured to determine a second measured value for the solids content from the measured second microwave signal, and/or
wherein the first measured value and the second measured value are included when detecting the inhomogeneity.

27. The measurement assembly according to claim 17,
wherein the first microwave antenna device is further configured to emit a third microwave signal having a third main radiation direction that differs from the first main radiation direction and the second main radiation direction,
wherein the measurement circuit is further configured to detect the inhomogeneity on the basis of the first microwave signal, the second microwave signal and a third microwave signal.

28. The measurement assembly according to claim 27,
wherein a measuring tube longitudinal section passing through the first microwave antenna device divides the measuring tube into a first measuring tube section and a second measuring tube section,
wherein the second main radiation direction points into the first measuring tube section, and the third main radiation direction points into the second measuring tube section.

29. The measurement assembly according to claim 17,
wherein the inhomogeneity includes asymmetric deposit formation, partial filling of the measuring tube, and/or sedimentation formation.

30. The measurement assembly according to claim 17,
wherein the first main radiation direction and the second main radiation direction lie in a common cross section of the measuring tube intersecting at least the first microwave antenna device.

31. The measurement assembly according to claim 17,
wherein the second main radiation direction and the third main radiation direction each has a directional component that points in a longitudinal direction of the measuring tube.

32. A method for determining any inhomogeneity in a measuring tube, comprising:
providing a measurement assembly, including:
a measuring tube for conveying the medium, wherein the measuring tube has a first antenna receptacle and a second antenna receptacle opposite the first antenna receptacle;
a first microwave antenna device arranged in the first antenna receptacle, wherein the first microwave antenna device is designed to emit a first microwave signal having a first main radiation direction and a second microwave signal having a second main radiation direction that differs from the first main radiation direction;
a second microwave antenna device that is arranged in the second antenna receptacle, wherein the second microwave antenna device is configured to measure the first microwave signal and the second microwave measurement signal; and
a measurement circuit, connected to the first microwave antenna device,
wherein the measurement circuit has a high-frequency generator for supplying the first microwave antenna device with the first microwave signal and the second microwave signal,
wherein the measurement circuit is configured to determine the property of the medium at least on the basis of a measured first microwave signal, and
wherein the measurement circuit is further configured to detect any inhomogeneity in the medium in the measuring tube on the basis of the measured first microwave signal and a measured second microwave signal;
emitting, via the first microwave antenna device, the first microwave signal having the first main radiation direction into the measuring tube;
receiving, via the second microwave antenna device, the first microwave signal after emission, wherein the received first microwave signal results at least from the emitted first microwave signal and its interaction with the medium;
emitting, via the first microwave antenna device, the second microwave signal having the second main radiation direction into the measuring tube, wherein the first main radiation direction differs from the second main radiation direction;
receiving the second microwave signal after emission via the second microwave antenna device, wherein the received second microwave signal results at least from the second microwave signal and its interaction with the medium, wherein the second microwave signal is reflected at least once on an inner lateral surface of the measuring tube so that the second propagation direction spatially changes at least once between the emission of the second microwave signal and the reception of the second microwave signal; and
detecting whether there is any inhomogeneity in the measuring tube depending on the received first microwave signal and the second microwave signal.
